(19) United States
(12) Patent Application Publication
(10) Pub. No.: US 2010/0305076 A1
(43) Pub. Date: Dec. 2, 2010

(54) METHODS OF TREATMENT FOR ULCERATIVE COLITIS

(76) Inventors: Chyon-Hwa Yeh, Cincinnati, OH (US); Linda Mary Law, Geneva (CH); Gino Regalli, Montgomery, OH (US); Nora Lee Zorich, Cincinnati, OH (US); Joan Marie Meyer, Wyoming, OH (US); Pamela Jean Schofield, Cincinnati, OH (US)

Correspondence Address: FITZPATRICK CELLA HARPER & SCINTO, 1290 Avenue of the Americas, NEW YORK, NY, 10104-3800, US

(21) Appl. No.: 12/309,930

(22) PCT Filed: Sep. 13, 2006
(86) PCT No.: PCT/US2006/035427

§ 371 Date: Aug. 12, 2010

(51) Int. Cl. A61K 31/606; A61P 1/04

(52) U.S. Cl. 514/166

(57) ABSTRACT

Disclosed herein are new treatments for moderate ulcerative colitis in various subject subgroups. The various subject subgroups include Caucasians, non-smokers, subjects under the age of about 65 years, and subjects previously or currently being treated with steroids.

### TECHNICAL FIELD

The present invention relates to methods of treatment for ulcerative colitis for various subject subgroups. These subgroups include Caucasians, non-smokers, subjects under the age of about 65 years, and subjects previously or currently being treated with steroids.

### BACKGROUND OF THE INVENTION

Ulcerative colitis (UC) is a condition that causes inflammation and sores in the form of ulcers, in the lining of the rectum and colon. The inflammation may kill the cells that line the colon, causing ulcers. Inflammation in the colon may also cause the colon to empty frequently, causing diarrhea. When the inflammation occurs in the rectum and lower part of the colon it is called ulcerative proctitis. If the entire colon is affected it is called pancolitis. If only the left side of the colon is affected it is called left-sided or distal colitis.

UC is a type of inflammatory bowel disease (IBD). IBD is the general name for diseases that cause inflammation in the small intestine and colon. UC is oftentimes difficult to diagnose as it shares symptoms common to other intestinal disorders and to Crohn's disease, another type of IBD. Crohn's disease differs because it causes inflammation deeper within the intestinal wall and can occur in other parts of the digestive system including the small intestine, mouth, esophagus, and stomach.

One known method of drug therapy to treat UC is administration of aminosalicylates. Aminosalicylates include 5-aminosalicylic acid (5-ASA), salts thereof, and pro-drugs that release 5-aminosalicyclic acid, or salts thereof, in vivo. Pro-drugs that release 5-aminosalicylic acid, or salts thereof, in vivo include, but are not limited to: olsalazine, balsalazide, and sulfasalazine. Aminosalicylates may be administered orally, through an enema, or in a suppository. Most people with mild or moderate ulcerative colitis are treated with aminosalicylates drugs first. The aminosalicylates also used in cases of relapse and to maintain remission.

ASACOL® is a product comprising the aminosalicylate, 5-aminosalicylic acid or mesalamine. ASACOL® is effective in treating patients with mild to moderate ulcerative colitis. Its effectiveness also extends to the maintenance of remission for prolonged periods. The current recommended dose of orally delivered ASACOL® for active disease is two 400-mg tablets three times daily for a total of 2.4 g/day (grams per day) for the treatment of mild to moderate UC. If the patient does not respond to ASACOL®, then alternatives, such as corticosteroids, are considered.

The inventors find herein that wherein the subject is a member of a specific subject subgroup and the UC is moderate, administration of mesalamine at doses of greater than about 2.4 g/day, or an amount of another aminosalicylate to deliver greater than about 2.4 g/day of 5-ASA, shows significant improvement in the condition in comparison to doses of 2.4 g/day of 5-ASA.

### BRIEF SUMMARY OF THE INVENTION

In one aspect of the present invention, there is a method of treating moderate ulcerative colitis in a human subject comprising the step of orally administering to the human subject an aminosalicylate in an amount to deliver to the subject more than about 2.4 g/day but less than or equal to about 4.8 g/day of 5-aminosalicylic acid, wherein the human subject is selected from the group consisting of human subjects under about 65 years of age, Caucasian human subjects, non-smoking human subjects; and, previous or current steroid-using mammalian subjects. In one embodiment, the step of orally administering an aminosalicylate comprises orally administering an aminosalicylate in an amount to deliver about 4.8 g/day of 5-aminosalicylic acid to the subject. In preferred embodiments comprising the delivery of about 4.8 g/day of 5-aminosalicylic acid to the subject, the aminosalicylate comprises mesalamine or a salt thereof. In some embodiments comprising the delivery of about 4.8 g/day of 5-aminosalicylic acid to the subject, the step of orally administering comprises orally administering once per day, twice per day, three times per day, or four times per day. In some embodiments, the aminosalicylate comprises mesalamine or a salt thereof. In some embodiments wherein the aminosalicylate comprises mesalamine or a salt thereof, the aminosalicylate comprises mesalamine and the mesalamine is administered in an amount of about 4.8 g/day. In some embodiments, the step of orally administering comprises orally administering tablets comprising about 800 milligrams of mesalamine or a salt thereof. In some embodiments comprising the oral administration of about 800 milligram tablets of mesalamine, the tablets are delayed-release tablets. In some embodiments, the step of orally administering comprises orally administering tablets comprising about 1.2 g mesalamine or a salt thereof. In some embodiments comprising the oral administration of about 1.2 g tablets of mesalamine, the tablets are delayed-release tablets. In some embodiments, the aminosalicylate comprises a component selected from the group consisting of mesalamine, a salt of mesalamine, olsalazine, a salt of olsalazine, balsalazide, a salt of balsalazide, sulfasalazine, a salt of sulfasalazine, or any pharmaceutically acceptable combination thereof. In some embodiments, the step of orally administering comprises orally administering once per day, twice per day, three times per day, or four times per day. In some embodiments, the human subject is male.

In another aspect of the present invention, there is a method of treating moderate ulcerative colitis in a human subject comprising the step of administering to the human subject an aminosalicylate in an amount to deliver to the subject more than about 2.4 g/day but less than or equal to about 4.8 g/day of 5-aminosalicylic, acid, wherein the human subject is selected from the group consisting of human subjects under about 65 years of age, Caucasian human subjects, non-smoking human subjects, and previous or current steroid-using mammalian subjects. In some embodiments, the step of administering comprises rectal administration. In one embodiment, the step of administering an aminosalicylate comprises administering an aminosalicylate in an amount to deliver about 4.8 g/day of 5-aminosalicylic acid to said subject. In preferred embodiments comprising the delivery of about 4.8 g/day of 5-aminosalicylic acid to the subject, the aminosalicylate comprises mesalamine or a salt thereof. In some embodiments comprising the delivery of about 4.8 g/day of 5-aminosalicylic acid to the subject, the step of administering comprises administering once per day, twice per day, three times per day, or four times per day. In some embodiments, the aminosalicylate comprises mesalamine or a salt thereof. In some embodiments wherein the aminosalicylate comprises mesalamine or a salt thereof, the aminosalicylate comprises mesalamine and the mesalamine is administered in an amount of about 4.8 g/day. In some embodiments, the step of administering comprises administering a rectal composition comprising about 800 milligrams or about 1.2 g of mesalamine or a salt thereof. In some embodiments comprising administration of a rectal composition, the rectal composition is an enema. In some embodiments, the rectal composition is a foamed composition. In some embodiments, the aminosalicylate comprises a component selected from the group consisting of mesalamine, a salt of mesalamine, olsalazine, a salt of olsalazine, balsalazide, a salt of balsalazide, sulfasalazine, a salt of sulfasalazine, or any pharmaceutically acceptable combination thereof. In some embodiments, the step of administering comprises administering once per day, twice per day, three times per day, or four times per day. In some embodiments, the human subject is male.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIG. 1 shows outcomes by demographic characteristics comparing a 2.4 g/day regime with a 4.8 g/day regime.

FIG. 2 shows outcomes by disease history comparing a 2.4 g/day regime with a 4.8 g/day regime.

FIG. 3 shows outcomes by baseline disease activity comparing a 2.4 g/day regime with a 4.8 g/day regime.

### DETAILED DESCRIPTION OF THE INVENTION

As used herein, “a” or “an” means one or more. Unless otherwise indicated, the singular contains the plural and the plural contains the singular.

As used herein, “aminosalicylate” refers to a class of compounds capable of releasing 5-amino-2-hydroxybenzoate or 5-amino-2-hydroxybenzoic acid as an active moiety in vivo. Non-limiting examples include mesalamine (5-amino-2-hydroxybenzoic acid), olsalazine (3,3′-dicarboxy-4,4′-dihydroxyazobenzene), balsalazide ((E)-5-[[4-[[(2-carboxyethyl)amino]carbonyl]phenyl]azo]-2-hydroxybenzoic acid), and sulfasalazine (2-hydroxy-5-[[4-[(2-pyridinylamino)sulfonyl]phenyl]azo]-benzoic acid). Although the examples provided describe the free acid, free amine forms, the term is not so limited and encompasses the free acid forms, the free amine forms, and any salts thereof. A composition comprising an aminosalicylate may have one or more than one aminosalicylate in addition to other possible components. The active moiety is illustrated below:

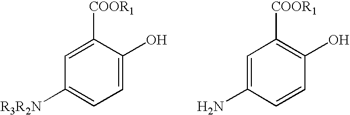

wherein R₁ can be hydrogen or a physiologically relevant counterion and the nitrogen can be further protonated and carry a positive charge along with a physiologically relevant counterion.

As used herein, “mesalamine” means 5-amino-2-hydroxybenzoic acid. The term “mesalamine” covers the free acid, the free amine, and any salts thereof. The term “mesalamine” may also be used interchangeably with “mesalazine”, “5-ASA” or “5-aminosalicylic acid”.

As used herein, the term “moderate” in relationship to ulcerative colitis will be commonly understood in the art and means a level of UC disease activity in which the subject exhibits rectal bleeding and colon wall friability, with an absence of or insignificant, systemic toxicity. Determination of moderate UC will therefore be consistent with Kornbluth et al., “Ulcerative colitis practice guidelines in adults (update) ACG”, Practice parameters committee, Am. J. Gastroenterol. 2004, 99: 1371-1385.

As used herein, the term “non-smoking subject” means a subject that does not smoke cigarettes, cigars, or the like concurrent with practice of the method herein.

As used herein, the term “previous or current steroid-using” with reference to the mammalian (e.g., human) subject under treatment means that the subject currently (i.e., concurrent with practice of the method herein) or previously has used (i.e., prior to practice of the method herein) a steroid therapy to treat ulcerative colitis.

As used herein, “treating” refers to the amelioration and/or delay of at least one symptom of a medical condition and in particular embodiments does not necessarily encompass a cure for the medical condition.

The inventors have found that administration of a weight of aminosalicylate to deliver more than about 2.4 g/day but less than or equal to about 4.8 g/day of 5-aminosalicylic acid (5-ASA) to a human subject of a specific subject subgroup having moderate Ulcerative colitis provides superior therapeutic benefits in comparison to the delivery of 2.4 g/day of 5-ASA that is typically provided to such subjects. In one embodiment, the route of administration is oral administration in the form of tablets. In one embodiment, the tablets are delayed-release tablets. However, other forms of administration, particularly rectal administration, also benefit from the new regimen and is therefore within the scope of the present invention. Where rectal administration is used, enemas or foamed composition are the preferred dosage form. The weight of 5-ASA administered is determined using the ratio of molecular weights of the aminosalicylate and the molecular weight of 5-ASA along with the number of moles of 5-ASA delivered by that aminosalicylate. When the aminosalicylate is mesalamine, the molecular weight ratio is unity and the weight administered is equal to the weight of 5-ASA delivered.

The inventors find herein that administration of mesalamine at about 4.8 g/day provides a clear efficacy benefit over the 2.4 g/day regimen and addresses an unmet medical need for specific human subject subgroups with moderate ulcerative colitis. The safety profile of this regimen is comparable to that of the 2.4 g/day regimen.

Data from subjects with mild to moderate UC are combined and analyzed from two phase III, multi-center, randomized, double-blind, controlled clinical trials of identical design assessing the safety and clinical efficacy of an increased dosage of 5-ASA.

The primary endpoint is the percentage of moderate UC subjects achieving overall improvement (i.e., treatment success) from baseline at week 6. This is defined as: (1) complete response (remission); complete resolution of signs and symptoms (stool frequency, rectal bleeding, Subject Functional Assessment (SFA) and sigmoidoscopy score) and a Physician's Global Assessment (PGA) of 0; or (2) partial response; improvement from baseline in the PGA score and improvement in at least one clinical assessment (stool frequency, rectal bleeding, SFA and sigmoidoscopy score) and no worsening in any of the other clinical assessments.

Results of the primary analysis in subjects with moderate disease remain statistically significant after adjustment for demographic or baseline characteristics using the Cochram-Mantel-Haenszel test stratified by subgroup variable.

Pre-specified subgroup analyses for fifty-four demographic and baseline characteristics are performed in subjects with moderately active disease (PGA score=2) to assess consistency of primary endpoint.

### Example 1

Analysis of the efficacy data in men with moderate disease demonstrates significant benefit from the 4.8 g/day regimen compared to the lower dose in this population in both studies, whether analyzed according to the pre-specified primary analysis or using set-to-failure (Table 1). The robustness of the results in men is supported by the consistency of the results for the primary analysis and for the set-to-failure analyses as shown in Table 1.

TABLE 1
Success Rates in Male Human Subjects with Moderate
Disease at Baseline.

| | | | | | Difference in | |
|---|---|---|---|---|---|---|
| | n | 2.4 g/day | N | 4.8 g/day | Proportions | p-value |
| Primary | | | | | | |
| Analysis | | | | | | |
| Study 1 | 58 | 50.0% | 53 | 75.5% | 25.5% | 0.0057 |
| Study 2 | 43 | 48.8% | 38 | 76.3% | 27.5% | 0.0111 |
| Set-to-Failure | | | | | | |
| Study 1 | 62 | 46.8% | 54 | 74.1% | 27.3% | 0.0028 |
| Study 2 | 44 | 47.7% | 40 | 72.5% | 24.8% | 0.0209 |

The results in men with moderate disease are consistent with the expected success rates used to design both studies. In designing these studies, the sample size is based on the following assumptions: the success rate for the 2.4 g/day treatment group would be 40% and the success rate for the 4.8 g/day treatment group would be 60%. Thus, the hypothesized true difference between treatment groups is 20%. Observed differences of approximately 25% in men are consistent with the hypothesized value.

The observed p-values and robustness of the data in men, sufficiently provide strong support for the use of a daily regimen of 4.8 g/day in men with moderate UC. Concerns about the effects of multiplicity on the significance and robustness of these results are mitigated by the fats that the overall primary analysis in Study 1 is statistically significant and provides similar consistent evidence from the moderately active study subpopulation and the subgroup of men.

In addition, a Bayesian analysis is conducted to calculate the probability that treatment with the 4.8 g/day dose in men results in a higher success rate than treatment with the 2.4 g/day does. This type of analysis considers data from any previous studies conducted using either of the two dose levels. Success rates from male subjects with moderate disease at baseline from three previous mesalamine studies are used to estimate the prior distributions for the 2.4 g/day dose and the 4.8 g/day dose. Using these prior distributions and Bayes analyses, the “posterior distribution” is calculated for each dose level based on the date obtained from previous studies.

The results from the Bayesian analysis show that the probability of a successful treatment outcome using the 4.8 g/day dose is 74.7% (95% credible interval: 64.9%, 83.5%). The interpretation of the 95% credible interval means that there is a 95% probability that the success rate for the 4.8 g/day dose is between 64.9% and 83.5%.

The probability of a successful treatment outcome in men using the 2.4 g/day dose is 47.2% (95% credible interval: 35.7%, 58.9%). There is a 95% probability that the success rate for the 2.4 g/day dose is between 35.7% and 58.9%. Finally, as shown graphically below, the probability that treatment with 4.8 g/day dose in men will result in a higher success rate than the 2.4 g/day dose is 99.97%. The analysis further supports the robustness of the results from the male population in the clinical program.

### Example 2

Evidence from the current studies supporting an additional benefit with the 4.8 g/day dose in women is present but is not as strong as that seen for men. The expected response rate in the 2.4 g/day group (as estimated for the purpose of study sizing using data from previous studies) is 40%. The actual response rate exceeded 60% (Table 2). In contrast to the results in men with moderate disease, for whom the 4.8 g/day regimen clearly provided additional benefit over the lower doses, the majority of women with moderate disease in these studies are adequately treated by the lower dose.

TABLE 2
Success Rates in Female Subjects with Moderate
Disease at Baseline.

| | | | | | Difference in | |
|---|---|---|---|---|---|---|
| | n | 2.4 g/day | N | 4.8 g/day | Proportions | p-value |
| Primary | | | | | | |
| Analysis | | | | | | |
| Study 1 | 72 | 66.7% | 71 | 69.0% | 2.3% | 0.7638 |
| Study 2 | 50 | 64.0% | 38 | 68.4% | 4.4% | 0.6647 |
| Set-to-Failure | | | | | | |
| Study 1 | 77 | 62.3% | 75 | about | 3.0% | 0.7008 |
| | | | | 65.3% | | |
| Study 2 | 52 | 62.5% | 44 | 59.1% | −2.4% | 0.8070 |

Exploratory analysis of Study 1 and Study 2 suggest that the 4.8 g/day (800 mg tablet) dose may provide additional benefit over the currently approved 2.4 g/day dose (400 mg tablet) in a subpopulation of women with moderate ulcerative colitis and more severe symptoms.

Analyses of Treatment Response in Women with Moderate Disease and Other Factors at Baseline:
Moderate Females only (i.e. PGA=2)
Steroid/Previous steroid use
For each baseline variable (bowel, blood, sigmoidoscopy), subject must have score of at least 2
Combined protocols and for each protocol separately
N is total number of females across both groups that met the criteria

TABLE 3

| | | | |
|---|---|---|---|
| | | 4.8 g/day | |
| | 2.4 g/day | Success | |
| N | Success Rate % | Rate (%) | p-value |

Primary endpoint results - Week 6 - Moderate Female Only

| | | | | |
|---|---|---|---|---|
| Baseline | | | | |
| Combinations | | | | |
| Combined (N = 248) | | | | |
| Steroid | 85 | 41 | 73 | .003 |
| Bowel and blood and | 73 | 51 | 76 | .03 |
| sig | | | | |
| Steroid or (bowel and | 129 | 46 | 74 | .002 |
| blood and sig) | | | | |
| Baseline | | | | |
| Combinations Study 1 | | | | |
| (N = 152) | | | | |
| Steroid | 52 | 43 | 79 | .008 |
| Bowel and blood and | 45 | 67 | 81 | .28 |
| sig | | | | |
| Steroid or (bowel and | 80 | 55 | 75 | .06 |
| blood and sig) | | | | |
| Baseline | | | | |
| Combinations Study 2 | | | | |
| (N = 96) | | | | |
| Steroid | 33 | 38 | about 65 | .12 |
| Bowel and blood and | 28 | 27 | 69 | .02 |
| sig | | | | |
| Steroid or (bowel and | 49 | 33 | 73 | .006 |
| blood and sig) | | | | |

Set to Failure Results - Week 6 - Moderate Female Only

| | | | | |
|---|---|---|---|---|
| Baseline | | | | |
| Combinations | | | | |
| Combined (N = 248) | | | | |
| Steroid | 92 | 38 | 67 | .007 |
| Bowel and blood and | 82 | 49 | 63 | .18 |
| sig | | | | |
| Steroid or (bowel and | 142 | 43 | about 65 | .01 |
| blood and sig) | | | | |
| Baseline | | | | |
| Combinations Study | | | | |
| 1 (N = 152) | | | | |
| Steroid | 55 | 41 | 73 | .02 |
| Bowel and blood and | 50 | 62 | 71 | .49 |
| sig | | | | |
| Steroid or (bowel and | 87 | 51 | 68 | .12 |
| blood and sig) | | | | |
| Baseline | | | | |
| Combinations Study | | | | |
| 2 (N = 96) | | | | |
| Steroid | 37 | 33 | 58 | .13 |
| Bowel and blood and | 32 | 27 | 53 | .13 |
| sig | | | | |
| Steroid or (bowel and | 55 | 31 | 62 | .02 |
| blood and sig) | | | | |

Notably, those women with a history of corticosteroid use to manage their ulcerative colitis and/or current active disease characterized by a score of 2 or greater (scale 0 to 3) in the rectal bleeding, stool frequency, and endoscopic scores of the disease, are more likely to benefit from the higher'dose (Table 4).

TABLE 4
Success Rates in Women with Moderate Disease
(PGA scores ≧2) and Other Factors at Baseline.

| | | | 4.8 | Difference in | |
|---|---|---|---|---|---|
| Primary Endpoint | n | 2.4 g/day | g/day | Proportions | p-value |
| Study 1 | | | | | |
| History of steroid use | 82 | 43% | 79% | 36% | 0.008 |
| Bowel and blood and | 45 | 67% | 81% | 14% | 0.28 |
| sigmoidoscopy scores | | | | | |
| each ≧2 | | | | | |
| History of steroid use or | 80 | 55% | 75% | 20% | 0.06 |
| bowel and blood and | | | | | |
| sigmoidoscopy each ≧2 | | | | | |
| Study 2 | | | | | |
| History of steroid use | 33 | 38% | about | 27% | 0.12 |
| | | | 65% | | |
| Bowel and blood and | 28 | 27% | 69% | 42% | 0.02 |
| sigmoidoscopy scores | | | | | |
| each ≧2 | | | | | |
| History of steroid use or | 49 | 33% | 73% | 40% | 0.006 |
| bowel and blood and | | | | | |
| sigmoidoscopy each ≧2 | | | | | |

### Example 3

Analysis of the efficacy data in specific subject subgroups with moderate disease demonstrates significant benefit from the 4.8 g/day regimen compared to the lower dose in the same populations. The subject subgroups include include age, race, steroid use, and smoking status.

A total of 687 subjects are randomized in Studies I and II, of which 423 analyzable subjects had moderately UC.

Among subjects with moderately UC, 4.8 g/day mesalamine (800 mg tablet) is superior to 2.4 g/day (400 mg tablet) for achieving overall improvement in subjects with moderately active UC (72% vs. 58%, p<0.05).

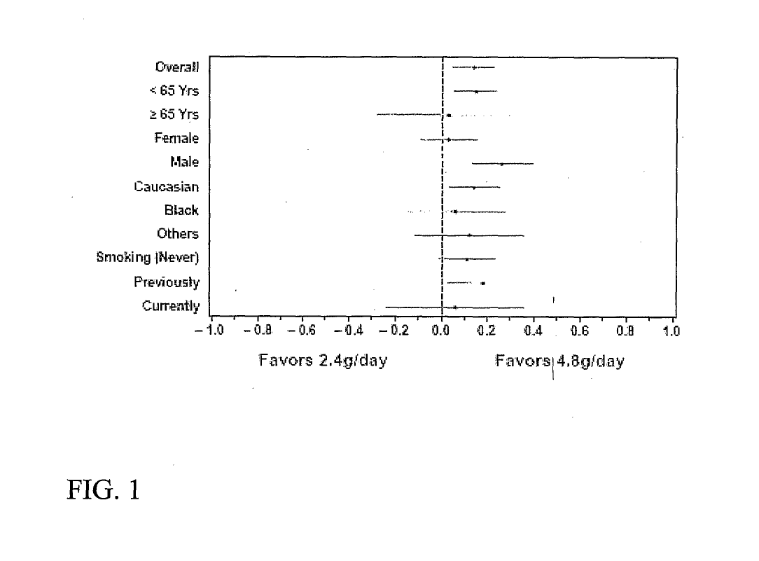

FIG. 1 shows outcomes by demographic characteristics. What is noteworthy from FIG. 1 is the increased benefit of a 4.8 g/day mesalamine therapy in comparison to a 2.4 g/day therapy for subjects under about 65 years of age, Caucasians, and non-smokers.

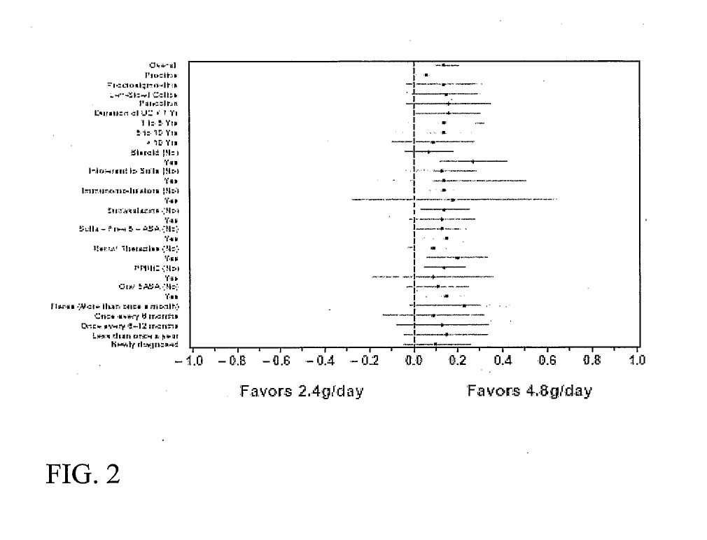

FIG. 2 shows outcomes by disease history. FIG. 2 shows that former steroid users enjoy increased therapeutic benefits using a 4.8 g/day mesalamine therapy in comparison to a 2.4 g/day therapy. The results show consistency across the multiple pre-specified subpopulation analyses within each study regardless of the analyses (i.e., set to failure) performed. To further evaluate the robustness of the results in subjects with moderate disease, whisker plots (i.e., point estimate and 95% confidence interval for the difference between the 2.4 g/day group and the 4.8 g/day group) for various subgroups defined on the basis of baseline characteristics (e.g., disease severity, demographic parameters) are prepared.

The results are defined on the basis of the following characteristics:
Age (<about 65 years, about 65 years)
Race (Caucasian, Black, Other)
Smoking (never, previously, currently)
Disease location (proctitius, left-sided colitis, pancolitis)
Duration of ulcerative colitis (<1 year, >1 year and ≧5 years, >5 years and ≦10 years, >10 years)
Drug history
   Use of steroids (yes/no)
   Intolerant to sulfa (yes/no)
   Use of immunomodulators (yes/no,
   Use of sulfasalazine (yes/no)
   Use of sulfa-free aminosalicylate (yes/no)
   Use of rectal therapies (yes/no)
   Use of PPI/H2 (yes/no)
   Use of oral 5-ASA (yes/no)
Frequency of flares (>1 per month, 1 per 6 months, 1 per 6-12 months, <>1 per year, newly diagnosed)

Each of these subgroups is pre-specified in the statistical analysis plan prior to unblinding the study. As can be seen from the point estimates and confidence intervals for the differences between the 4.8 g/day group and the 2.4 g/day group, the 4.8 g/day group is consistently superior to the 2.4 g/day group, with many of these differences (51 of 54 subgroups for males with moderate disease in the combined population) being significantly favorable to the 4.8 g/day group.

The data demonstrate that the following subject subgroups show benefit from a 4.8 g/day dosage of 5-ASA in comparison to the traditional 2.4 g/day dosage:
Subjects under about 65 years of age;
Caucasian subjects;
Non-smokers;
Previous or current steroid users.

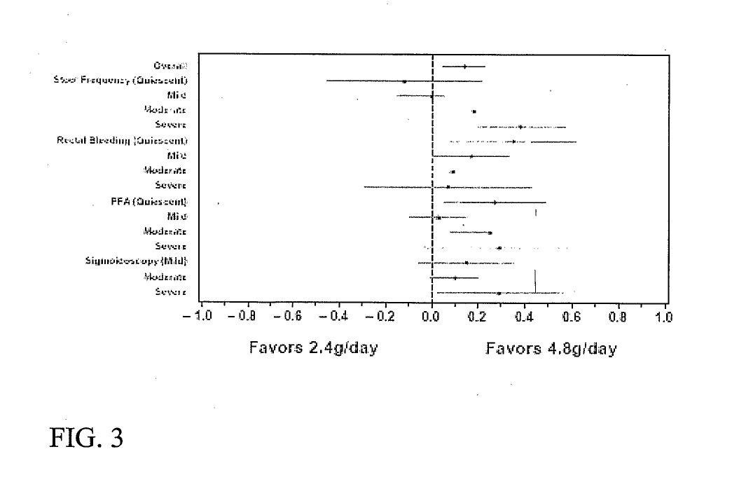

FIG. 3 shows outcomes by baseline disease activity. Subjects having moderate UC consistently show increased therapeutic benefits using a 4.8 g/day mesalamine therapy in comparison to a 2.4 g/day therapy.

The results in the specific subject subgroups with moderate disease are consistent with the expected success rates used to design both studies. In designing these studies, the sample size is based on the following assumptions: the success rate for the 2.4 g/day treatment group would be 40% and the success rate for the 4.8 g/day treatment group would be 60%. Thus, the hypothesized true difference between treatment groups is 20%. Observed differences of approximately 25% in men are consistent with the hypothesized value.

Table 5 provides the combined subgroup analysis showing overall improvement at week 6 for subjects with moderate UC. The 4.8 g/day dose (800 mg tablet) of mesalamine is well tolerated with adverse events comparable to the 2.4 g/day (400 mg tablet) dose.

TABLE 5
Combined Subgroup Analysis.

| | 2.4 g/day n = 23 | 4.8 g/day n = 200 |
|---|---|---|
| Previous Steroid Use | | |
| Yes | 52% | 79% |
| No | 62% | 69% |
| Disease Extent | | |
| Proctitis | 71% | 77% |
| Proctosigmoiditis | 56% | 71% |
| Left-sided colitis | 56% | 71% |
| Pancolitis | 55% | 71% |
| Gender | | |
| Male | 50% | 76% |
| Female | 66% | 69% |

### Example 4

A 70 kg non-smoking man diagnosed with moderate Ulcerative colitis is prescribed a pharmaceutical oral composition comprising 1.2 g of olsalazine (mol. wt. 302.24), a mesalamine dimer, two tablets to be taken twice daily for a total of 4.8 g/day of the 5-ASA dimer(5-ASA mol. wt.=153.14). The subject takes two tablets of the pharmaceutical in the morning and two tablets in the evening such that about 4.8 g/day of 5-ASA is delivered. The Physician's Global Assessment (PGA) score improves in comparison to baseline and rectal bleeding reduces.

Molecular weights and the moles of 5-ASA delivered per mole of aminosalicylate are used to determine the amount of 5-ASA delivered when the aminosalicylate to be administered is one other than mesalamine. Complete cleavage of pro-drug forms is assumed. For example, for the aminosalicylate olsalazine (OLSAL), the following equation is used to determine the approximate weight of olsalazine needed to deliver a targeted amount of about 4.8 g of 5-aminosalicylic acid:

(4.8 g 5-ASA)*(1 mole 5-ASA/153.14 g 5-ASA)*(1 mole OLSAL/2 mole 5-ASA)*(302.24 g OLSAL/1 mole OLSAL)=4.7 g OLSAL; wherein * signifies multiplication.

Using a twice daily regimen:

(4.7 g OLSAL/2 times per day)=2.4 g OLSAL at each time per day, which can be administered in the form of two tablets containing 1.2 g of OLSAL in the morning and evening.

Molecular weights and in the number of moles of 5-ASA delivered per mole of aminosalicylate are shown in Table 6 below for some illustrative aminosalicylates.

TABLE 6
Molecular Weights and Moles of 5-ASA Delivered for Some
Aminosalicylates.

| | Moles of 5-ASA per | |
|---|---|---|
| Aminosalicylate (as free | mole of | |
| acid) | Aminosalicylate | Molecular weight |
| Mesalamine | 1 | 153.14 |
| Balsalazide | 1 | 357.32 |
| Olsalazine | 2 | 302.24 |
| Sulfasalazine | 1 | 398.40 |

### Example 5

A 60 kg woman diagnosed with moderate ulcerative colitis and previously treated with steroid therapy is prescribed a pharmaceutical composition comprising four 1.2 g mesalamine delayed-release tablets, to be taken once daily. The four tablets are taken in the morning so that 4.8 g/day of 5-ASA is delivered. The Physician's Global Assessment (PGA) score improves in comparison to baseline and rectal bleeding is reduced.

### Example 6

A 75 kg Caucasian man diagnosed with moderate ulcerative colitis is prescribed a pharmaceutical oral composition comprising two 1.2 g balsalazide delayed-release tablets, to be taken three times daily for a total of 7.2 g/day of balsalazide; this regimen is calculated to deliver about 3.1 g of 5-ASA The Physician's Global Assessment (PGA) score improves in comparison to baseline and rectal bleeding is reduced.

### Example 7

A fifty year old, 71 kg man diagnosed with moderate Ulcerative colitis is prescribed a pharmaceutical composition comprising a rectal mesalamine foam. The foam is administered three times per day (morning, afternoon, and evening) such that 1 g of mesalamine is administered at each interval for a total of 3 g of mesalamine per day. The Physician's Global Assessment (PGA) score improves in comparison to baseline and rectal bleeding is reduced.

Example 7 provides an embodiment of the present invention using non-oral administration of aminosalicylate. Non-limiting suitable examples of a rectal composition are described in U.S. Pat. No. 5,082,651 issued to Healy et al. on Jan. 21, 1992.

Aminosalicylate treatment may be used to deliver weights of 5-ASA which are greater than the prior art 2.4 g/day, up to and including a daily dosage of about 4.8 g/day. This range includes delivered dosages of 2.5 g/day, 2.6 g/day, 2.7 g/day, 2.8 g/day, 2.9 g/day, 3.0 g/day, 3.1 g/day, 3.2 g/day, 3.3 g/day, 3.4 g/day, 3.5 g/day, 3.6 g/day, 3.7 g/day, 3.8 g/day, 3.9 g/day, 4.0 g/day, 4.1 g/day, 4.2 g/day, 4.3 g/day, 4.4 g/day, 4.5 g/day, 4.6 g/day, 4.7 g/day, and 4.8 g/day of aminosalicylate (e.g., 5-ASA), as well as numerical values in-between the stated dosages. In certain embodiments the aminosalicylate is 5-ASA. In certain embodiments, the delivered dosage is 4.8 g/day of 5-ASA.

While the certain administration comprises administration of three daily doses, the administration may comprise other schedules, such as, but not limited to, administration once per day, administration twice per day, administration three times per day, and administration four times per day.

### CONCLUSIONS

Among subjects with moderate UC, overall improvement with a 4.8 g/day delayed-release oral mesalamine (800 mg tablet) is consistent across a number of subject subgroups. The incremental benefit of a 4.8 g/day over 2.4 g/day is more apparent in men than in women and more apparent in subjects previously treated with steroids. No baseline or demographic characteristics predict treatment failure. Both 4.8 g/day delayed-release oral mesalamine (800 mg tablet) and 2.4 g/day (400 mg tablet) are well tolerated and had similar safety profiles.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the composition of matter, and methods described in the specification. As one of ordinary skill in the art will readily appreciate from the disclosure of the present invention, compositions of matter, methods, or steps, presently existing or later to be developed that perform substantially the same function or achieve substantially the same result as the corresponding embodiments described herein may be utilized according to the present invention. Accordingly, the appended claims are intended to include within their scope such processes, compositions of matter, methods, or steps.

What is claimed is:

1. A method of treating moderate ulcerative colitis in a mammalian subject comprising he step of administering to said mammalian subject an aminosalicylate in an amount to deliver to said subject more than about 2.4 g/day of 5-aminosalicylic acid, wherein said mammalian subject is selected from the group consisting of:
human subjects under about 65 years of age,
Caucasian human subjects,
non-smoking human subjects; and,
previous or current steroid-using mammalian subjects.

2. The method of claim 1, wherein the mammalian subject is a human.

3. The method of claim 2, wherein said step of administering an aminosalicylate comprises orally administering an aminosalicylate in an amount to deliver less than about 4.8 g/day of 5-aminosalicylic acid to said subject.

4. The method of claim 3, wherein said aminosalicylate comprises mesalamine or a salt thereof.

5. The method of claim 3, wherein said step of orally administering comprises orally administering once per day, twice per day, three times per day, or four times per day.

6. The method of claim 2, wherein said aminosalicylate comprises mesalamine or a salt thereof.

7. The method of claim 6, wherein said aminosalicylate comprises mesalamine and further wherein said mesalamine is administered in an amount of about 4.8 g/day.

8. The method of claim 2, wherein said step of orally administering comprises orally administering a tablet comprising about 800 milligrams of mesalamine or a salt thereof.

9. The method of claim 8, wherein said tablet is a delayed-release tablet.

10. The method of claim 2, wherein said aminosalicylate comprises a component selected from the group consisting of mesalmine, salts of mesalamine, olsalazine, salts of olsalazine, balsalazide, salts of balsalazide, sulfasalazine, salts of sulfasalazine, and combinations thereof.

11. The method of claim 2, wherein said step of orally administering comprises orally administering once per day, twice per day, three times per day, or four times per day.

12. The method of claim 2, wherein the human subject is under about 65 years of age.

13. The method of claim 2, wherein the human subject is Caucasian.

14. The method of claim 2, wherein the human subject is a non-smoking human subject.

15. The method of claim 2, wherein the human subject is a previous or current steroid user.

16. The method of claim 2, wherein the step of administering comprises rectal administration.

17. The method of claim 16, wherein said step of administering an aminosalicylate comprises administering an aminosalicylate in an amount to deliver about 4.8 g/day of 5-aminosalicylic acid to said subject.

18. The method of claim 17, wherein said aminosalicylate comprises mesalamine or a salt thereof.

19. The method of claim 18, wherein said step of administering comprises administering once per day, twice per day, three times per day, or four times per day.

20. The method of claim 16, wherein said aminosalicylate comprises mesalamine or a salt thereof.

21. The method of claim 16, wherein said aminosalicylate comprises mesalamine and further wherein said mesalamine is administered in an amount of about 4.8 g/day.

22. The method of claim 16, wherein said step of administering comprises administering a rectal composition comprising about 800 milligrams or about 1.2 g of mesalamine or a salt thereof.

23. The method of claim 16, wherein said rectal composition is an enema.

24. The method of claim 16, wherein said rectal composition is a foam.

25. The method of claim 16, wherein said aminosalicylate comprises a component selected from the group consisting of mesalamine, salts of mesalamine, olsalazine, salts of olsalazine, balsalazide, salts of balsalazide, sulfasalazine, salts of sulfasalazine, and combinations thereof.

26. The method of claim 16, wherein said step of administering comprises administering once per day, twice per day, three times per day, or four times per day.

27. The method of claim 16, wherein the human subject is under about 65 years of age.

28. The method of claim 16, wherein the human subject is Caucasian.

29. The method of claim 16, wherein the human subject is a non-smoking human subject.

30. The method of claim 16, wherein the human subject is a previous or current steroid user.
